(19) United States
(12) Patent Application Publication
(10) Pub. No.: US 2007/0121067 A1
(43) Pub. Date: May 31, 2007

(54) INTRAOCULAR PRESSURE AND BIOMECHANICAL PROPERTIES MEASUREMENT DEVICE AND METHOD

(76) Inventor: Andrew Peter Davis, Bellevue, WA (US)

Correspondence Address: BLACK LOWE & GRAHAM, PLLC, 701 FIFTH AVENUE, SUITE 4800, SEATTLE, WA, 98104, US

(21) Appl. No.: 11/563,662

(22) Filed: Nov. 27, 2006

Related U.S. Application Data
(60) Provisional application No. 60/739,541, filed on Nov. 26, 2005.

(51) Int. Cl. A61B 3/10

(52) U.S. Cl. 351/212

(57) ABSTRACT

A measurement probe for measuring interocular and biomechanical properties pressure is provided. The probe includes an array sensor associated with one end of a housing for application to a corneal area such that an area smaller than the diameter of the array sensor is applanated when area calculations are made and force measurements are taken. A computer component is in communication with the array sensor such that data is received and recorded from the array sensor. A display is provided for displaying the data associated with the interocular pressure and biomechanical properties taken using the array sensor. A methodology is also provided for determining the interocular pressure and biomechanical properties associated with a corneal area.

### PRIORITY CLAIM

This application claims priority to U.S. Provisional Application Nos. 60/739,541, filed Nov. 26, 2005, which application is hereby incorporated by reference in its entirety as if fully set forth herein.

### FIELD OF THE INVENTION

This invention relates generally to intraocular pressure measurement and, more specifically, to a device and method that uses thin-film force and pressure sensors to measure the intraocular pressure and biomechanical properties of the cornea.

### BACKGROUND OF THE INVENTION

The instrument that is the gold standard of intraocular pressure measurement is the Goldmann applanation tonometer, which was initially described in 1957. This device determines intraocular pressure (IOP) by measuring the force needed to flatten a circle with a diameter of 3.06 mm at the central cornea. It has an optical endpoint that is used to determine when this diameter is reached. This instrument is almost 50 years old, but it still is the standard against which all other instruments are compared. Although there were early tonometers that held the force constant and varied the surface area to measure the IOP, most tonometers today vary the force needed to applanate a fixed area.

There are particular shortcomings of the Goldmann tonometer and its progeny. First, by requiring an optical endpoint, error and bias are introduced into the measurement. Moreover, additional equipment, namely, a slit lamp must used to take the IOP measurement. The quality of the tear film can also influence the reading. Any corneal disease that affects the corneal surface can make it difficult to take a reading. Finally, there has been a recent growth of interest in how the biomechanical properties of the cornea can also affect the reading. The corneal thickness has been found to vary considerably, and can falsely elevate or reduce the measurement by several mmHg. Concern is also growing that other biomechanical properties of the cornea can be just as important in influencing the measurement.

Other tonometers have been developed to try to improve on the shortcomings of the Goldman applanation tonometer. One such tonometer, the “Tono-Pen,” uses a strain gauge on a small plunger to measure force needed to applanate a small fixed area of the plunger tip, using a logic circuit to determine when the pressure tracing dips, indicating the cornea is then being flattened by the area surrounding the plunger. Although the tonopen has no optical endpoint it tends to give readings that can be highly variable. The pneumotonometer is similar to the tonopen, except that the sensor is reading the pressure of compressed air used to control the plunger. The noncontact tonometer uses a puff of air to deform the cornea, and then measures the time required to flatten the cornea, detected when the light is reflected in a particular way which only happens when the corneal apex has been flattened. Typically, noncontact tonometers are highly inaccurate and are used as screening tools. A recent version of the noncontact tonometer, The Corneal Response Analyzer, takes two pressure measurements, one when the cornea is moving in, and one when the cornea is moving out, and uses this difference as a measure of the overall resistance of the cornea or the hysteresis of the cornea. Another tonometer recently introduced is the Dynamic Contour Tonometer uses a tiny strain gauge sitting in a curved housing that measures the IOP by measuring the force at the gauge when the corneal curvature matches the curvature of the housing. The validity and accuracy of this method has yet to be established.

Within the past two years, there has been the development of thin-film force and pressure sensors, such as described by Tekscan, Inc. in South Boston, Mass. These array sensors can identify both the amplitude of force and the location of the force. An array sensor is produced by a matrix of intersecting rows and columns of printed electrodes, with an additional layer of semiconductor ink providing electrical resistance at each intersection. When force is applied, the change of resistance at each location can be measured and displayed graphically. There have been sensors made with special resolution as fine as 0.0229 mm2. Such a matrix sensor measures both a static and dynamic footprint of pressure distribution.

Accordingly, there is a need for an improved device and method for IOP measurement that overcome the errors and other limitations associated with prior measurement devices and procedures. The present invention describes a new and improved device and method that uses thin-film force and pressure sensors to measure the IOP as well as new methods to measure and analyze the biomechanical properties of the cornea.

### SUMMARY OF THE INVENTION

The present invention provides a measurement probe for measuring interocular pressure. In a preferred embodiment, the measurement probe including an array sensor associated with one end of a housing for application to a corneal area such that an area smaller than the diameter of the array sensor is applanated when interocular pressure measurements are taken. A computer component is in communication with the array sensor such that data is received and recorded from the array sensor. A display is provided for displaying the data associated with the interocular pressure measurements taken using the array sensor.

In alternative embodiments, methods are provided for determining the interocular pressure and biomechanical properties associated with a corneal area. In a preferred embodiment, an array sensor associated with one end of a housing is applied to a corneal area such that an area smaller than the diameter of the array sensor is applanated. The area of corneal contact with the array sensor is determined. The force measurements associated with the corneal area of contact with the array sensor are obtained. Next, depending on the application, one or both of the interocular pressure and biomechanical properties, including error induced by biomechanical forces during applanation, is calculated using the area data and force measurements. The resulting area data and force measurements—interocular pressure, biomechanical properties data or both—are displayed.

### BRIEF DESCRIPTION OF THE DRAWINGS

Preferred and alternative embodiments of the present invention are described in detail below with reference to the following drawings.

FIG. 1 is a cutaway side view of an IOP measurement probe made in accordance with the invention;

FIG. 2 is a graphical illustration of the relationship of the total average force needed to applanate a certain area of the cornea using a preferred device and method of the present invention;

FIG. 3 is a graphical illustration of the relationship between the IOP and the diameter of applanation using a preferred device and method of the present invention;

FIG. 4 is an illustration of an exemplar static footprint of applanation for a specific average force using a preferred device and method of the present invention;

FIG. 5 is an illustration of a partial three dimensional exemplar static footprint of applanation for a specific average force using a preferred device and method of the present invention;

FIG. 6 is an illustration of a three dimensional exemplar static footprint of applanation for a specific average force using a preferred device and method of the present invention;

FIG. 7 is an illustration of an exemplar cross-sectional profile of the application footprint shone in FIG. 6; and

FIG. 8 is a process flow diagram of a method for measuring interocular pressure and biomechanical properties using the device of the present invention.

### DETAILED DESCRIPTION OF THE PREFERRED EMBODIMENT

The present invention describes a new and improved device and method to measure the IOP and analyze the biomechanical properties of the cornea. The device and method uses thin-film force and pressure to overcome the errors and other limitations associated with prior measurement devices and procedures.

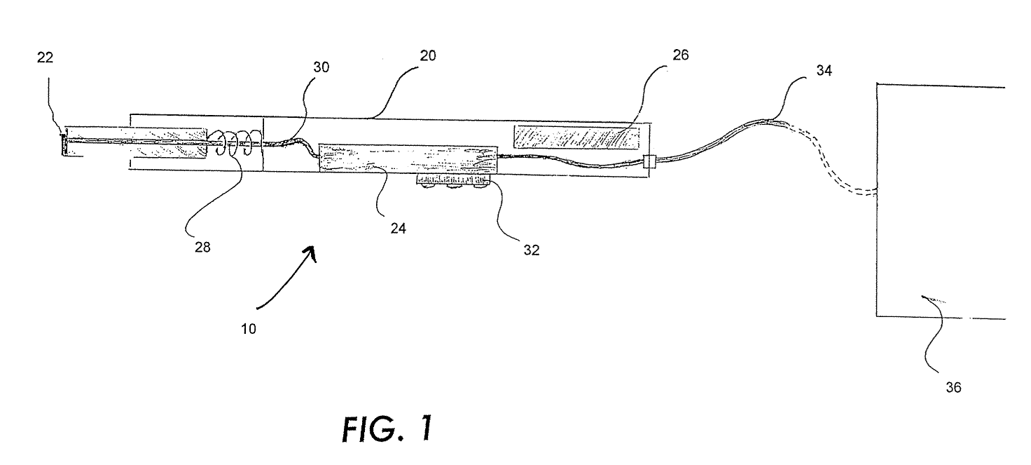

A preferred measurement probe 10 is described with reference to FIG. 1. The device 10 includes a housing 20, an array sensor 22 reciprocally associated to an end of the housing 20, a microprocessor circuit/multiplexing hardware component 24 and a digital display 26 for displaying the results of the measurement and other related data. A spring assembly 28 is preferably connected between the array sensor 22 and the housing 20 to provide for movement of the array sensor as it contacts with a corneal area. Alternative embodiments include designs without a spring. A first wire assembly 30 connects the array sensor 22 to the microprocessor circuit/multiplexing hardware component 24. A power reset switch 32 is preferable used to control the power to the measurement probe 10. In a preferred embodiment, a second wire assembly 34 is used to connect the measurement probe 10 to an independent computing device 36 that may be used to record, evaluate and/or disseminate data obtained from the array sensor 22. In an alternative embodiment, many of the functions associated with the independent computer device 36 may be incorporated into the housing 20.

In operation in association with IOP measurements, the measurement probe 10 is turned on and the housing is gently applied so that the array sensor contacts the cornea. The contact is preferably in a manner such that an area smaller than the diameter of the sensor is flattened (applanated). The measurement probe 10 is then gently removed. The probe 10 can signal when a certain threshold force or area is reached. The spring allows a more gradual increase and decrease of force and helps to avoid excessive force on the cornea.

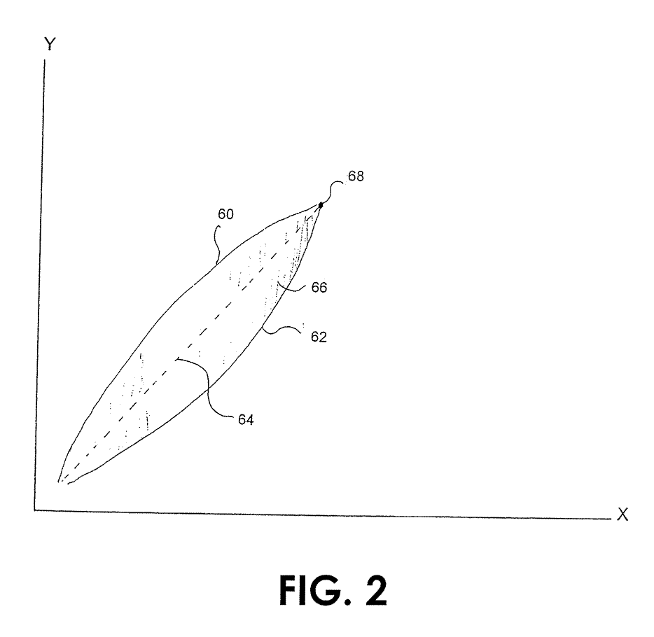

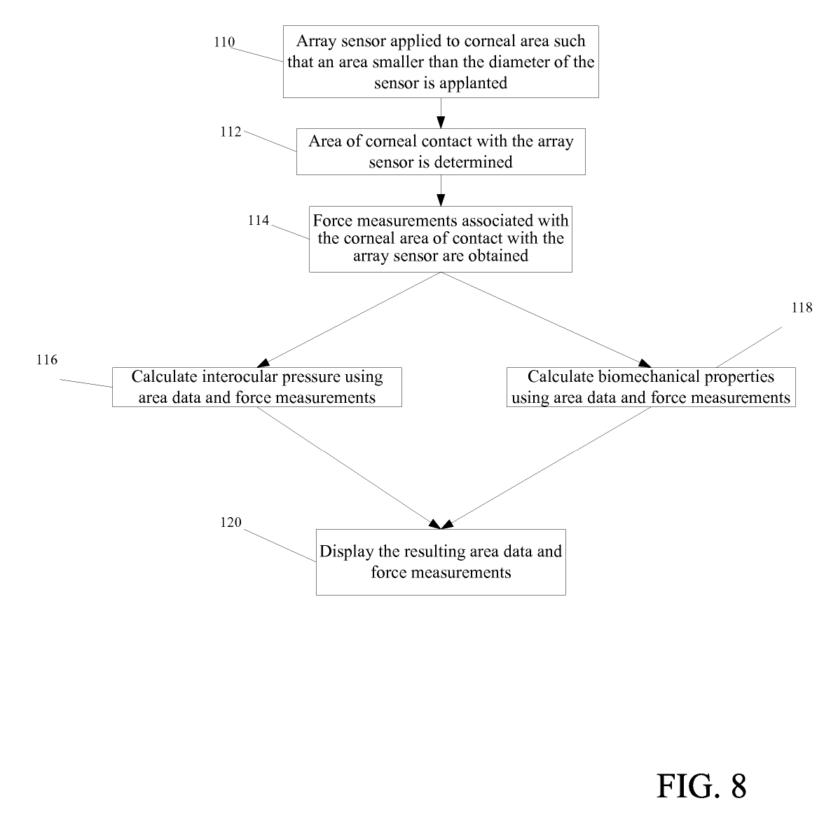

The measurement probe 10 of the present invention can be used to measure IOP using various methodologies that are described with reference to FIGS. 2-7, and described generally with reference to FIG. 8. At block 110, an array sensor associated with one end of a housing is applied to a corneal area such that an area smaller than the diameter of the array sensor is applanated. At block 112, the area of corneal contact with the array sensor is determined. At block 114, force measurements associated with the corneal area of contact with the array sensor are obtained. Next, depending on the application, one or both the blocks 116 and 118 may occur. At block 116, the interocular pressure is calculated using the area data and force measurements. At block 118, biomechanical properties, including error induced by biomechanical forces during applanation, is calculated. At block 120, the resulting area data and force measurements—interocular pressure, biomechanical properties data or both—are displayed.

In a first embodiment, as the measurement probe 10 is pressed lightly against the cornea the footprint of contact enlarges as the pressure increases on the probe. A real time special and amplitude analysis can then be performed. Using the independent computer device 36, the area of contact and the average force in the area of contact for each point in time according to the sampling rate is calculated. This relationship can be displayed graphically, as shown with reference to FIG. 2, where the x-axis is the area of applanation and the y-axis is the total average force. Curve 60 represents the relationship of the total average force needed to applanate a certain area as the probe 10 is applied to the cornea (measured in “down-step”). Curve 62 represents the relationship between force and area as the probe 10 is removed from the cornea (measured in “up-step”). The average slope of either curve estimates the IOP. Curve 64 is the average between curve 60 and curve 62 and represents the calculated equilibrium. The average slope of curve 64 represents a more accurate calculation of the IOP. Additionally, the area 66 between curves 60 and 62 represents a more comprehensive assessment of corneal hysteresis than has been possible with prior measurement methodologies. Point 68 represents a point of equilibrium, and can also be used to calculate the IOP. Alternative methods to analyze IOP and corneal hysteresis include graphing the total average force as a function of the diameter of applanation, graphing the calculated IOP as a function of the area of applanation, and graphing the calculated IOP as a function of the diameter of the area of applanation in both down-step and up-step.

In a second embodiment, the data collected by the measurement probe 10 can be analyzed in a different way to calculate the IOP and corneal rigidity. The force required to deform the cornea, distinct from the force needed to applanate the cornea against the IOP, is related to both the diameter of the area applanated and to the corneal rigidity and thickness. The error induced by biomechanical forces during applanation increases as the size of the applanation area increases. This relationship is amplified by the biomechanical properties of the cornea. In other words, for a large applanation diameter, the rigid cornea shows far more error than the flexible cornea, whereas at smaller area of applanation the difference in error is less significant. Graphing the calculated IOP as a function of the diameter of applanation area (or as a function of the area of applanation) allows for another means to ascertain the biomechanical error. Without this biomechanical error, the graph yields horizontal lines, with a constant IOP independent from the diameter of applanation.

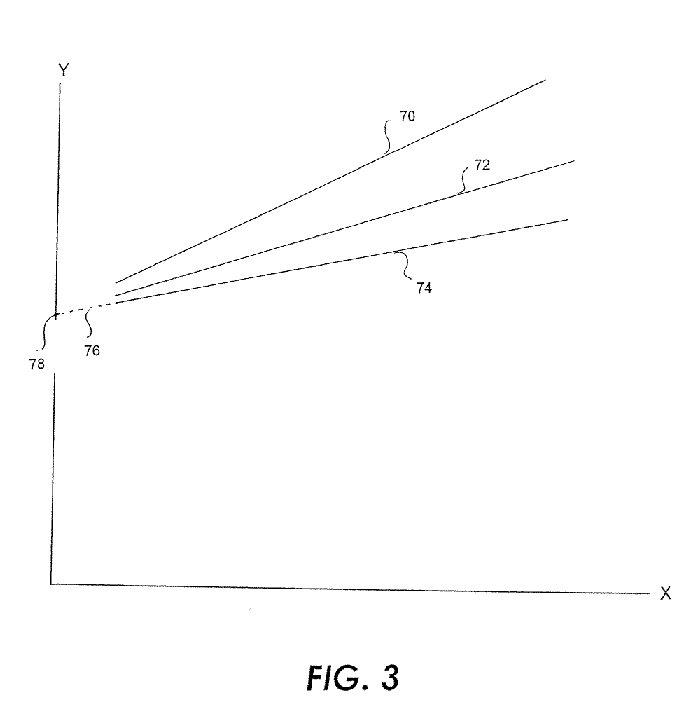

FIG. 3 is a graphical illustration of the relationship between the IOP and the diameter of applanation. In FIG. 3, the x-axis is the diameter of area of applanation and the y-axis is the IOP. Lines 70, 72 and 74 represent three different corneas with different biomechanical properties. Line 70 represents a thick inflexible cornea with more induced biomechanical error while line 74 represents a thin flexible cornea with less induced biomechanical error. The average slope of the IOP compared to the diameter (or surface area) curve is used to calculate the elasticity/rigidity factor, and the measured IOP adjusted accordingly. Additionally, it is possible to extrapolate the curve 76 to estimate the IOP when the diameter (or surface area) approaches 0 (point 78 in FIG. 3). The above analysis can be made used when looking at the IOP curves in up-step, down-step, or calculated equilibrium.

If computational hardware and software become too complex to perform a continuous read out of force and surface area as the values are changing, shortcuts in both the hardware and software can be used to look at only several points along the curve. For example, the probe 10 may be used to take readings when the surface area reaches three arbitrary numbers, or to measure the surface areas at three pre-selected average forces.

In yet an alternative of this embodiment, the probe 10 could be used to take a single reading at a pre-selected force, or a pre-selected surface area. This single reading is preferably measured in up-step and in down-step. The resulting average is used to calculate IOP and the difference between the up-step and down-step measurements is used to estimate the corneal hysteresis.

The measurement probe 10 is not affected by the capillary forces of the tear film. Because the applanation area is the sensor array, it is the net force that is being measured, which already includes the contribution of the tear film. By contrast, in other tonometers, the force is measured distant from the cornea interface and outside the influence of the tear film. As a result, the effect of the tear film must be subtracted. In yet other embodiments of the measurement device, a condom-like coverings may be used over the array sensor 22 to obviate the need for accounting for the forces of the tear film. Regardless whether a condom like protector sleeve is used, the measurement probe 10 does not need to account for errors induced by the tear film. Additionally, errors induced by astigmatic error are also eliminated, since the area actually contacted is measured, whether or not it is a circle or an oval (the area applanated is an oval when the cornea has significant astigmatic error).

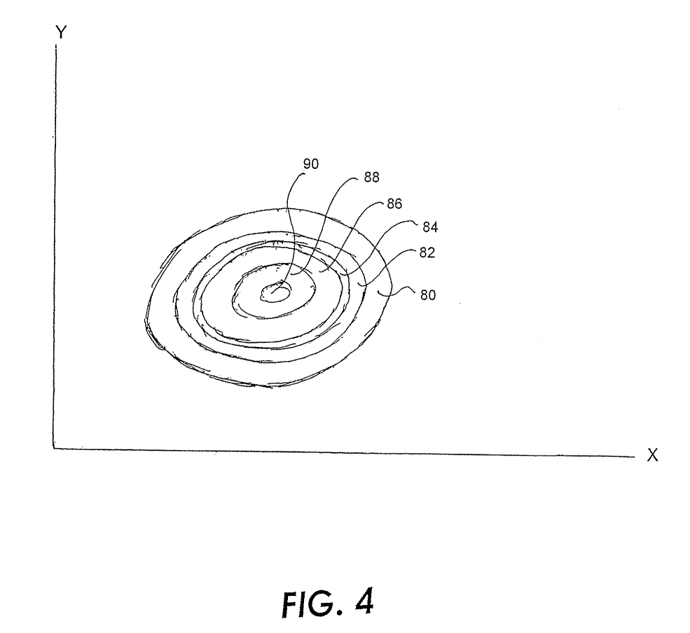

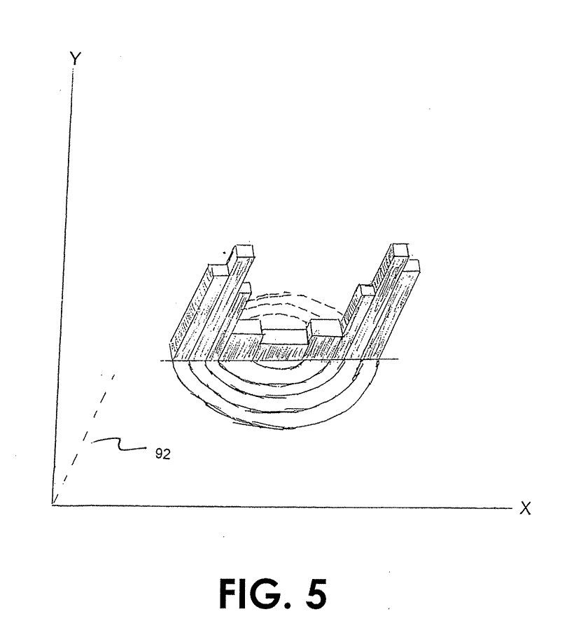

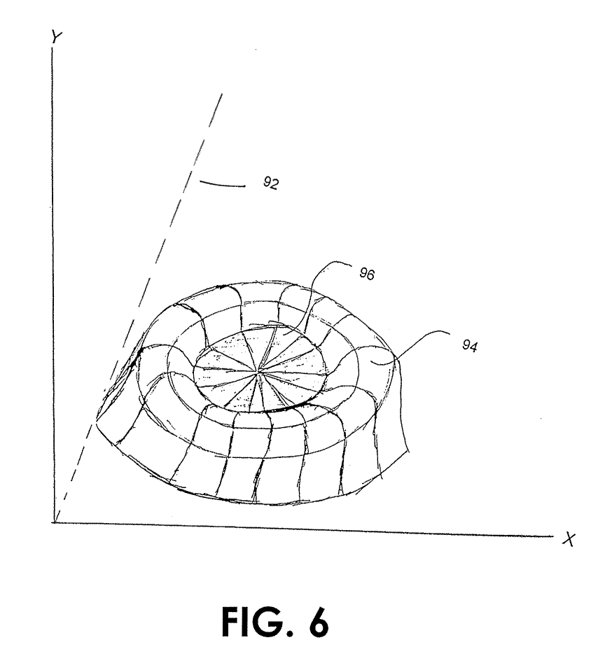

In a third embodiment, the measurement probe 10 can also be used to measure and calculate the IOP, as well as taking a direct measurement of the cornea's biomechanical properties, by analyzing the “footprint” of forces measured. Unlike prior tonometers that measure the total average force for the area applanated, the measurement probe 10 of the present invention enables visualization of the distribution of forces in the area applanated. FIG. 4 shows an example of a static footprint of applanation for a specific average force (or for a specific surface area). Both the x- and y-axes plot the location of the sensor array. The concentric circles represent isopters of force (or computed pressure). Areas 80-90 represent distribution of forces within a certain ranges. FIG. 5 shows a partial three-dimensional representation of the distribution of forces on the sensor array, with direction 92 representing force. FIG. 6 shows a fully three-dimensional representation of the distribution of forces on the sensor array. Direction 92 again represents force. In this example, the resulting measurement illustrates a bowl shape typical to the results of the measurement probe 10. The forces in the wall of the bowl (94 in FIG. 6) are higher than the floor of the bowl (96 in FIG. 6) because it is at this outer wall area that additional force is needed to “buckle” the cornea, changing its shape from round to flat. It is exactly these forces that induce the biomechanical error of applanation. In a theoretical, ideal, thin, elastic sphere, the footprint would simply be a flat plateau.

Analyzing this footprint enables several computations. For example, the IOP can be calculated by using the total surface area applanated and average force of the floor of the bowl (96 in FIG. 6).

In another example, the additional force represented by the rim of the bowl (94 in FIG. 6) can be calculated, thereby taking a direct measurement of the biomechanical aspects of the cornea which accounts for the error. These forces are included in the measurement regardless whether they relate to corneal thickness or other intrinsic flexibility factors. The IOP can be adjusted based on these measurements relating to the biomechanical factors. In other words, the forces needed to flex the cornea can be measured and subtracted from the forces needed to applanate the cornea against the intraocular pressure.

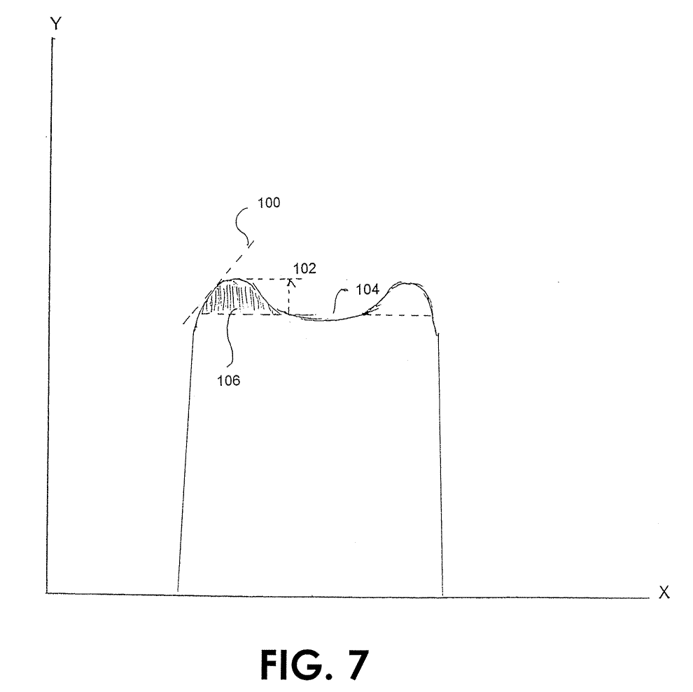

FIG. 7 represents a cross-sectional profile of the applanation footprint of FIG. 6, where the x-axis is the location and the y-axis is the force. The slope of the wall of the applanation footprint 100, the height of the wall 102 compared to the baseline in the bowl 104, or the area of the wall 106 can be used to estimate the biomechanical sources of error, which can then be used to calculate a corrected IOP. The volume of the wall (94 in FIG. 6) can also be used.

In another example, analysis of the dynamic applanation footprint allows for an even more sophisticated methodology to analyze the biomechanical forces. Looking at the area or volume of the wall as the area of applanation increases allows for the actual measurement of the sum of all the biomechanical sources of error encountered in reaching the measurement for a given surface area. Additionally, studying the changes in the shape of the wall as the applanation area is increased adds significant information about the biomechanical qualities of the cornea.

In yet another example, analysis of the static or dynamic applanation footprint provides data related to corneal hysteresis. The analysis of the footprint of applanation as discussed above was more directed at looking at data in down-step, or at the point of equilibrium. By evaluating the data obtained when the applanation footprint is taken in up-step, as well as in calculated equilibrium, further useful information is obtained. In looking at the applanation footprint in up-step, it is the unbuckling of the cornea caused by elastic qualities which contributes to the biomechanically induced error. Analyzing the difference from the baseline is thus a measure of the biomechanical elasticity of the cornea (whereas in down-step the difference relates more to biomechanical rigidity). It is the sum of both the biomechanical rigidity and elasticity which accounts for the total corneal hysteresis, and thus the total biomechanically induced error when applanating the cornea. Thus, looking at the differences from baseline in both up-step and down-step allows for measurement of the different components of the biomechanical qualities of the cornea, i.e. the measurement of the rigidity component in down-step, and the elasticity component in up-step.

In yet an alternative embodiment, the measurement probe 10 and methodology allows for measurement of the pulse pressure. In this embodiment, the measurement probe 10 is held by a means that a steady force can be applied to the tip of the probe containing the sensor array 22 against the cornea. One such means would be a balanced device mounted at a slit lamp. Another means would be a measurement probe 10 with a tip that rests against the cornea under its own weight. In an alternative embodiment, a spring type device is incorporated that allows for free movement of the probe array sensor 22 once a certain force is reached, without increasing the force. (It is not necessary to know what the steady force is, but only that it stays steady even as the tip is moved.) When the force of the measurement probe 10 against the cornea is held constant, the probe can measure the changes of surface area occurring with each pulse, and calculate the pulse pressure.

In yet another embodiment, the measurement probe 10 allows for IOP measurement through the eyelid. This embodiment provides advantages associated with home tonometry. In this embodiment, the measurement probe 10 is applied to the eyelid over the cornea. The footprint of applanation through the lid produces a different appearance than through the cornea. However, such differences are taken into consideration when calculating the biomechanical error induced by the lid.

While the preferred embodiment of the invention has been illustrated and described, as noted above, many changes can be made without departing from the spirit and scope of the invention. For example, while association of an independent computing device 36 allows for sophisticated analysis of the data from the measurement probe 10, it is not essential to many of the embodiments. If a particular parameter or analysis, as described in the preceding methodology, is deemed useful, these can be incorporated in the microprocessor circuit/multiplexing hardware component 24 or slit lamp mounted base (not shown), and displayed directly on a small screen or readout, without the need of an external computer. In other words, many of the embodiments can easily be self-contained and display selected calculations. In yet an alternative embodiment, the measurement probe 10 may be used to measure, display and evaluate biomechanical properties of the corneal area using the above-described methodology independent of the interocular pressure. Accordingly, the scope of the invention is not limited by the disclosure of the preferred embodiment. Instead, the invention should be determined entirely by reference to the claims that follow.

The embodiments of the invention in which an exclusive property or privilege is: claimed are defined as follows:

1. An apparatus for measuring interocular pressure, comprising:
a housing;
an array sensor associated with one end of the housing for application to a corneal area such that an area smaller than the diameter of the array sensor is applanated when interocular pressure measurements are taken;
a computer component for receiving data obtained by the array sensor; and
and a display for displaying the data associated with the interocular pressure measurements taken using the array sensor.

2. The apparatus of claim 1, wherein the housing is a hand-held device.

3. The apparatus of claim 1, wherein the array sensor is reciprocally associated with the housing to provide for movement of the array sensor as it contacts with a corneal area.

4. The apparatus of claim 1, wherein the computer components is fixed to the housing.

5. The apparatus of claim 1, wherein the data obtained by the array sensor is transmitted to a second computer component independent of the apparatus.

6. A method for determining the interocular pressure associated with a corneal area, comprising:
applying an array sensor associated with one end of a housing to a corneal area such that an area smaller than the diameter of the array sensor is applanated;
determining the area of corneal contact with the array sensor;
obtaining force measurements associated with the corneal area of contact with the array sensor;
calculating the interocular pressure using the area of corneal contact and force measurements; and
displaying data related to the area of corneal contact and force measurements associated with the applanation.

7. The method of claim 6, further comprising calculating error induced by biomechanical forces during applanation using the area of corneal contact and force measurements.

8. The method of claim 6, wherein the data related to the area of corneal contact and force measurements associated with the applanation is graphically displayed.

9. A method for determining the biomechanical properties associated with a corneal area, comprising:
applying an array sensor associated with one end of a housing to a corneal area such that an area smaller than the diameter of the array sensor is applanated;
determining the area of corneal contact with the array sensor;
obtaining force measurements associated with the corneal area of contact with the array sensor;
calculating error induced by biomechanical forces during applanation using the area of corneal contact and force measurements; and
displaying data related to the area of corneal contact and force measurements associated with the applanation.

10. The method of claim 6, further comprising calculating the interocular pressure using the area of corneal contact and force measurements.

11. The method of claim 9, wherein the data related to the area of corneal contact and force measurements associated with the applanation is graphically displayed.
